(19) United States
(12) Patent Application Publication
(10) Pub. No.: US 2024/0197161 A1
(43) Pub. Date: Jun. 20, 2024

(54) DEPTH RENDERING SCOPE

(71) Applicant: Welch Allyn, Inc., Skaneateles Falls, NY (US)

(72) Inventors: Gene J. Wolfe, Pittsford, NY (US); Joshua Hess, Moravia, NY (US)

(73) Assignee: Welch Allyn, Inc., Skaneateles Falls, NY (US)

(21) Appl. No.: 18/391,367

(22) Filed: Dec. 20, 2023

Related U.S. Application Data
(60) Provisional application No. 63/434,013, filed on Dec. 20, 2022.

(51) Int. Cl. A61B 1/227; A61B 1/00; A61B 1/04; G02B 30/27; G06T 15/00; H04N 13/305; H04N 23/50

(52) U.S. Cl. CPC A61B 1/227; A61B 1/00032; A61B 1/00042; A61B 1/00048; A61B 1/00055; A61B 1/04; G02B 30/27; G06T 15/00; H04N 13/305; H04N 23/555

(57) ABSTRACT

Various scopes and methods for generating and outputting a 3D image of a surface are described herein. An example method includes generating depth data by detecting a distance between at least one imager and a surface; generating 2D image data by detecting light reflected from the surface; generating a 3D image of the surface by combining the depth data and the 2D image data; and outputting the 3D image of the surface.

### CROSS-REFERENCE TO RELATED APPLICATION

This application claims priority to U.S. Provisional App. No. 63/434,013, which is titled “Depth Rendering Scope” and was filed on Dec. 20, 2022, and is incorporated by reference herein in its entirety.

### TECHNICAL FIELD

This application relates to generating and displaying a three-dimensional (3D) medical image. In some cases, a handheld device generates and displays the 3D medical image.

### BACKGROUND

Otoscopes are devices that illuminate and image the interior of a patient's ear. In many cases, otoscopes are handheld devices operated by clinicians, such as physicians or nurses. In general, an otoscope can be equipped with a disposable adapter configured to hold the otoscope to the patient's ear during use. Otoscopes are important devices to diagnose patients in a variety of clinical settings, such as emergency rooms, pediatric offices, and general practice clinics.

Most otoscopes are monocular devices with a single screen. Using the single screen, otoscopes an provide a two-dimensional (2D) image of the ear canal and its contents. For instance, an otoscope can be used to visualize the eardrum. As an alternative to conventional otoscopes, clinicians can visualize ear canal using a binocular microscope in conjunction with a speculum disposed in the patient's ear. The binocular microscope can be used to visualize the ear canal in three dimensions and potentially with enhanced illumination, but has a number of drawbacks that make it less convenient than conventional otoscopes. For example, the binocular microscope must be used while the patient is in a supine position while their head is lifted, which can be inconvenient for both the patient and the clinician. It can be difficult, for instance, to position pediatric patients for a binocular microscope. Furthermore, the binocular microscope is generally heavier and more expensive than a conventional otoscope.

Due to the inconvenience of binocular microscopes, monocular otoscopes are the dominant tool for visualization of the inner ear. However, studies have shown that reliance on monocular otoscopes to diagnose ear diseases results in more than a 50% chance of misdiagnosis, as compared to binocular microscopic otoscopy. Thus, there is a need for an otoscope that provides a 3D view of the inner ear, but that is lightweight, inexpensive, and easy to use.

### SUMMARY

Various implementations of the present disclosure relate to a handheld instrument that can obtain and display a 3D image of a subject. The instrument, for example, can be a dermascope, a fundascope, an otoscope, a nasoscope, anoscope, proctoscope, or an endoscope.

According to various cases, the instrument images a 3D surface of a subject. In some implementations, the instrument captures a 2D colorized image of the surface. For example, the instrument includes an imager including a 2D array of light sensors configured to detect the color and intensity of light reflected from the surface. Further, the instrument may capture a depth image of the surface of the subject. The instrument, for instance, utilizes optical coherence tomography (OCT), ultrasound, or some other depth imaging technique to generate the depth image. In various cases, the instrument generates a 3D image of the surface by combining the 2D image of the surface and the depth image of the surface. In some examples, the instrument generates a 3D mesh of the surface using the depth image, and then colorizes and/or texturizes the 3D mesh using the 2D image.

Further, the instrument displays the 3D image. In some cases, the instrument includes a lenticular display that includes a single 2D screen. The lenticular display can output the 3D image using an array of lenses that direct light output by the screen into different directions towards the two eyes of a user. As a result, the user can perceive the displayed image in three dimensions.

### DESCRIPTION OF THE FIGURES

The following figures, which form a part of this disclosure, are illustrative of described technology and are not meant to limit the scope of the claims in any manner.

FIG. 1 illustrates an example environment for generating and displaying a 3D image of a subject.

FIG. 2 illustrates an example environment in which a lenticular display outputs a 3D image to a viewer.

FIG. 3 illustrates an example environment for generating a 3D image of a subject using the same imager to obtain a 2D image and a depth image of the subject.

FIGS. 4A and 4B illustrate example resolutions of images generated by a depth rendering scope.

FIG. 5 illustrates an example scope configured to obtain and display a 3D image of a subject.

FIG. 6 illustrates an example lenticular display divided into three radial zones.

FIG. 7 illustrates an example of a depth image and a 2D image being combined to generate a 3D image of a surface.

FIG. 8 illustrates an example process for providing a 3D image of a surface.

FIG. 9 illustrates an example process for generating a 3D image of a surface.

FIG. 10 illustrates at least one example device configured to enable and/or perform the some or all of the functionality discussed herein.

### DETAILED DESCRIPTION

Various implementations of the present disclosure will be described in detail with reference to the drawings, wherein like reference numerals present like parts and assemblies throughout the several views. Additionally, any samples set forth in this specification are not intended to be limiting and merely set forth some of the many possible implementations.

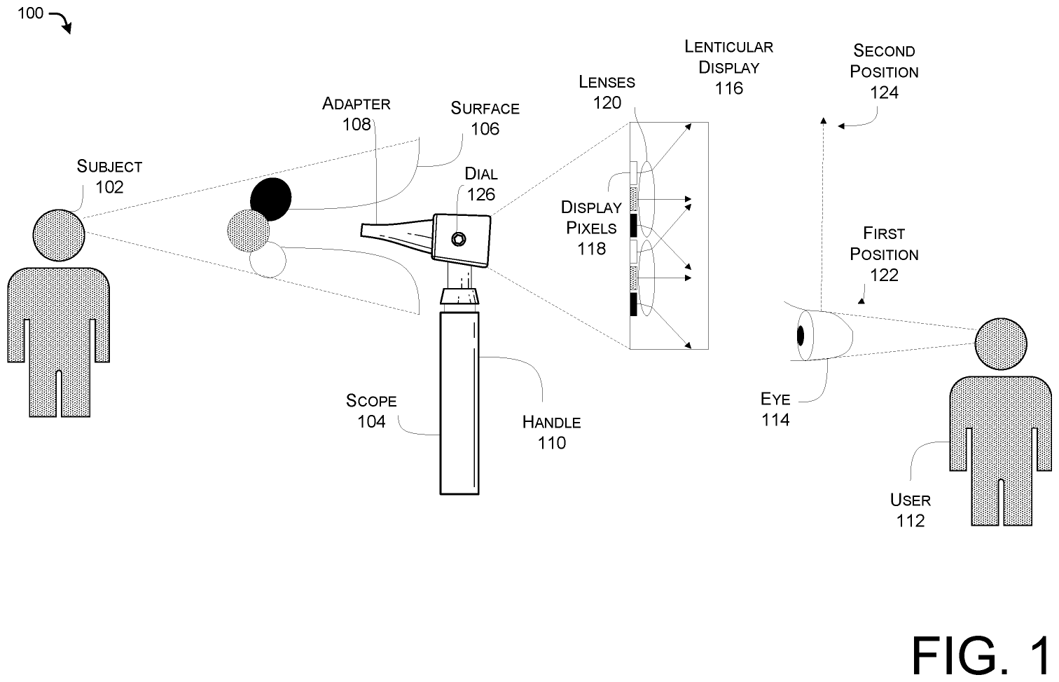

FIG. 1 illustrates an example environment 100 for generating displaying a 3D image of a subject 102. The subject 102, for instance, is a human, or another type of animal. According to some examples, the subject 102 is a patient in a clinical environment. For instance, the subject is being examined within a health clinic or hospital.

The environment 100 includes a scope 104. In various implementations, the scope 104 is an imaging device configured to image a surface 106 of the subject 102. In some examples, the surface 106 is an external surface of the subject. For instance, the surface 106 may be an outer surface of the skin of the subject 102. In some cases, the surface 106 is an interior surface of an orifice of the subject 102. For example, the surface 106 is an interior surface of a nostril, a mouth, an ear canal, an anus, or a vagina of the subject. According to some cases, the scope 104 includes an adapter 108 configured to be inserted into the orifice of the subject 102. In various implementations, the surface 106 is an interior surface of the subject 102, such as an interior surface of an abdominal cavity, a blood vessel, a heart, or a gastrointestinal tract. According to various implementations, the surface 106 can be planar or any curved surface that can be modeled as a tessellated mesh. The scope 104, for instance, is a dermascope, a fundascope, an otoscope, a nasoscope, anoscope, proctoscope, or an endoscope.

In various implementations, the scope 104 is portable. For example, the scope 104 may be equipped with one or more batteries that supply power to the scope 104 during operation. In some cases, the one or more batteries are rechargeable. For instance, the one or more batteries are configured to be recharged wirelessly in the presence of an electromagnetic field, or recharged via a wire configured to connect the scope 104 to mains power (e.g., via a port in the scope 104).

The elements of the scope 104, according to some examples, are enclosed in a housing. In some examples, the housing is waterproof. For instance, the housing includes a plastic shell that at least partially encloses the elements of the scope 104. In various cases, the scope 104 is configured to be hand-held. For instance, the housing of the scope 104 includes a handle 110 configured to be grasped by a user 112 during operation. The scope 104, for example, is relatively lightweight and is configured to be held by a single hand of the user 112. For instance, the scope 104 may weigh less than 2 kilograms (kg). The user 112, according to various cases, is a clinician. For instance, the user 112 is a physician, a nurse, a medical student, a medical technician, a resident, or a fellow. For instance, the user 112 may be examining the subject 102 with the scope 104 during a medical appointment. In various cases, the scope 104 is utilized in a primary care context. For instance, in some cases, the user 112 may be a family medicine practitioner, a nurse practitioner, a physician's assistant, or an internal medicine practitioner rather than a specialist.

In some cases, the scope 104 captures a two-dimensional (2D) image of the surface 106 of the subject. For example, the scope 104 emits light toward the surface 106 and detects the light reflected from the surface. According to some cases, the scope 104 includes at least one image sensor configured to generate the 2D image of the surface 106. In various implementations, the 2D image includes a 2D array of image pixels. As used herein, the term “image pixel,” and its equivalents, can refer to a unit of data that represents an area of a 2D image. In some implementations, the 2D image is referred to as “2D image data.” An example image sensor includes a 2D array of light sensors, wherein each individual light sensor is configured to generate an individual image pixel in the 2D image of the surface 106. For instance, the image sensor includes a single photon avalanche diode (SPAD), a charge-coupled device (CCD), an active-pixel sensor (APS), or any combination thereof. The image pixels of the 2D image may respectively correspond to the intensity and/or frequency of light reflected from different portions of the surface 106 and detected by the scope 104. In some examples, the 2D image is a grayscale image wherein each image pixel corresponds to a value between a minimum value and a maximum value, the value corresponding to the intensity of light received by the corresponding light sensor. In some instances, the 2D image is a color image wherein each image pixel corresponds to a set of values respectively corresponding to the intensities of a set of base colors of the light received by the corresponding light sensor. For example, the 2D image is a red-green-blue (RGB) image, wherein each image pixel is a set of three numbers corresponding to the intensity of red light, green light, and blue light components in the light received by the corresponding light sensor.

However, the 2D image may omit clinically significant details of the surface 106. For example, the surface 106 may include a bump or other three-dimensional (3D) structure that is indicative of a particular medical diagnosis. For example, if the subject 102 has a foreign body in their ear canal, but the foreign body is the same color and apparent texture of the ear canal, it may be difficult for the user 112 to identify the foreign body using the 2D image alone. Thus, it may be advantageous for the scope 104 to capture 3D information about the surface 106 in addition to the 2D image.

In various implementations of the present disclosure, the scope 104 generates a depth image representing the surface 106. For example, the scope 104 includes an ultrasound transducer configured to emit ultrasound toward the surface 106, to detect a reflection of the ultrasound from the surface 106, and to generate a depth image representing the distance between the surface 106 and the scope 104 based on the detected ultrasound. In some implementations, the depth image is also referred to as “depth image data.”

In some cases, the scope 104 generates the depth image using optical coherence tomography (OCT). For instance, the scope 104 may emit low-coherence light toward the surface 106 and detect an interference pattern including a reflection of the low-coherence light from the surface 106 and a reference beam of the low-coherence light. As used herein, the term “low-coherence light,” and its equivalents, may refer to a group of photons that are transmitted together and which have at least two frequencies. For instance, the scope 104 may include a halogen bulb, a light-emitting diode (LED), or a tungsten filament configured to generate the low-coherence light. In various cases, the scope 104 omits a laser that would otherwise generate coherent light. The scope 104 may generate the depth image based on the interference pattern. In various implementations, a single imager is configured to detect the 2D image and the interference pattern, which can reduce the size, weight, and complexity of the scope 104. For example, the same image sensor used to generate the 2D image may also be used to generate the depth image using OCT.

According to various examples, the scope 104 generates a 3D image of the surface 106 based on the 2D image and the depth image. For example, the scope 104 may texturize and/or colorize the depth image using the 2D image. According to some instances, the 2D image and the depth image have different resolutions. In various implementations, the scope 104 interpolates values of the 2D image onto locations of the surface 106 indicated by the depth image. The 3D image, in various implementations, includes voxels defined in three dimensions. As used herein, the term “voxel,” and its equivalents, may refer to a unit of data representing the intensity and/or frequency of light representing a unit of volume in a 3D image. In some cases, voxels with nonzero values in the 3D image correspond to the location distribution of the surface 106. These nonzero values, in various implementations, correspond to the color and/or intensity of the light reflected from the surface 106 along the location distribution. In some implementations, voxels representing volumes in the 3D image that do not align with the surface 106 are assigned zero values. According to some examples, the 3D image can be referred to as “3D image data.”

In some cases, the scope 104 is equipped with a single screen to display the 2D image, the depth image, or the 3D image. However, the single screen may include display pixels that are arranged two-dimensionally. In cases where the scope 104 includes two screens, the scope 104 can portray the 3D image to the user 112 by generating two derived 2D images that are projected from the 3D image at two different locations. The locations, for instance, can correspond to the perspective of two eyes 114 of the user 112. If the two screens respectively display the two derived 2D images to the respective eyes 114 of the user 112, the user 112 may perceive the 3D image due to the parallax induced from the two derived 2D images.

However, in some cases, it may be disadvantageous for the scope 104 to include two screens respectively displaying images to the eyes 114 of the user 112. For instance, the two screens can greatly increase the cost, size, weight, and energy consumption of the scope 104. Further, in some cases, the user 112 may seek to view the 3D image at a distance from the scope 104. For example, it may be cumbersome for the user 112 to bring her face up to the scope 104 in order to view the 3D image.

In various implementations of the present disclosure, the scope 104 displays the 3D image using a lenticular display 116. According to examples of the present disclosure, the lenticular display 116 includes a 2D array of display pixels 118 and an array of lenses 120. As used herein, the term “display pixels,” and their equivalents, can refer to light-emitting elements of a display screen. For example, the display pixels 118 may include light-emitting diodes (LEDs), organic LEDs (OLEDs), cold cathode fluorescent lamps, quantum dot (QD) display elements, thin-film transistors, or other types of light-emitting elements. The display pixels 118 are configured to emit light through at least some of the lenses 120.

The array of lenses 120 is disposed (e.g., overlaid) on the display pixels 118, such that the lenses 120 are disposed between the eye 114 of the user 112 and the display pixels 118. In various implementations, the lenses 120 are configured to transmit and/or refract light. The lenses 120 include a material transmissible to visible light. For example, the lenses 120 include glass, quartz, sapphire, or a transparent plastic. In some cases, the lenses 120 are spherical lenses. In various cases, the lenses 120 include Fresnel lenses.

As shown in FIG. 1, an individual lens 120 overlaps a group of the display pixels 118. According to some cases, the individual lens 120 refracts light from at least one first display pixel 118 in the group in a first direction to a first position 122. Further, the individual lens 120 refracts light from at least one second display pixel 118 in the group in a second direction to a second position 124. The first direction is different than the second direction. Accordingly, if the first display pixel 118 is controlled to emit light corresponding to a first voxel of the 3D image, and the second display pixel 118 is configured to emit light corresponding to a second voxel of the 3D image, then the lenticular display 116 can display the 3D image when viewed from the first position 122 and the second position 124. The scope 104, in various cases, controls the display pixels 118 such that the 3D image of the surface 106 is apparent from the first position 122 and the second position 124.

In various implementations, the user 112 can adjust the lenticular display 116 using an input device, such as a dial 126 on the side of the scope 104. For example, the dial 126 can be used to power on the scope 104, to adjust the brightness of the lenticular display 116, to adjust the brightness of the light source used to illuminate the surface 106, or the like.

The scope 104, in some examples, may display the 2D image, the depth image, the 3D image, or a combination thereof, on the lenticular display 116. In various cases, the scope 104, upon detecting an input signal via the dial 126, may cause the lenticular display 116 to switch between displaying the 2D image and the 3D image. For example, the user 112 may prefer to view the 2D image and operate the dial 126 accordingly.

In some implementations, the lenticular display 116 is associated with a predetermined viewing distance and viewing angles. For example, the lenses in the lenticular display 116 are configured to output the 3D image at a particular distance from the scope 104 at predetermined viewing angles, such that the user 112 can perceive the 3D image at the particular distance and in a position corresponding to the viewing angles. According to various examples, the scope 104 may be configured to detect whether the user 112 is disposed at the particular distance from the scope 104. For example, the scope 104 includes a distance sensor directed toward the eye 114 and which can detect a distance between the lenticular display 116 and the eye 114. Examples of the distance sensor include an ultrasound transducer, an infrared distance sensor, or a camera. For instance, the distance sensor is configured to generate a signal indicative of the distance between the lenticular display 116 and the eye 114 by detecting a time at which an ultrasound pulse takes to travel from the scope 104 to the user 112 and back again; by analyzing a reflection of an infrared pattern from the user 112; or by analyzing an image of the face of the user 112 that depicts the eye 114. In various implementations, the scope 104 detects a direction by which the eye 114 is disposed with reference to a surface of the lenticular display 116. For instance, the scope 104 includes a camera configured to detect an image of the eye 114, at least one processor within the scope 104 is configured to identify a pupil of the eye 114 depicted in the image, and the processor(s) detect the position of the eye 114 based on the identified depiction of the pupil.

In some examples, the scope 104 compares the detected distance to a predetermined viewing distance. In some cases, the scope 104 compares the position of the eye 114 to a predetermined viewing position range, as defined by the predetermined viewing angles. If the scope 104 determines that the detected distance is different than the predetermined viewing distance (e.g., outside of a predetermined range including the predetermined viewing distance), then the scope 104 may perform one or more remedial actions. Further, if the scope 104 determines that the position of the eye 114 is outside of the predetermined viewing position corresponding to the predetermined viewing angles, the scope 104 may perform the remedial action(s). In some examples, the one or more remedial actions include outputting an alert to the user 112 recommending that the user 112 adjust their position. In some cases, the alert is output as a visual alert on the lenticular display 116 and/or via a light integrated with the housing of the scope 104. In some implementations, the alert is output as an audible alert by a speaker within the scope 104. The alert, for instance, indicates a recommendation to increase or decrease the distance between the user 112 and the scope 104. In some implementations, the one or more remedial actions include dimming or deactivating the lenticular display 116 until the scope 104 detects that the distance between the lenticular display 116 and the eye 114 (or the position of the eye 114) has entered the predetermined range. In some cases, the one or more remedial actions include adjusting the lenses 120 to change the predetermined range. For instance, the scope 104 may be configured to alter the targeted viewing distance by tilting the lenses 120, moving the array of lenses 120 closer or farther from the display pixels 118, or the like. In some cases, the scope 104 includes one or more actuators configured to adjust the position of the lenses 120. In some examples, the scope 104 controls the display pixels 118 based on the detected distance (or viewing angle) between the lenticular display 116 and the eye 114.

The scope 104 described with reference to FIG. 1 is therefore configured to capture a 3D image of the subject 102 and output the 3D image to the user 112 using a single 2D array of display pixels 118. In various implementations, the scope 104 is a lightweight device that can be operated wirelessly. Accordingly, the scope 104 may be a convenient tool for the user 112 to utilize to assess a condition of the subject 102. In addition, the scope 104 can visually convey the surface 106 of the subject 102 in 3D, therefore capturing potentially critical information to the user 112 for accurately diagnosing a condition of the subject 102.

Although not specifically illustrated in FIG. 1, in some implementations, the scope 104 is adapted for wearable use. For example, the scope 104 can be integrated into a head-mounted display, such a pair of smart glasses. In some implementations, the lenticular display 116 is substituted for two displays configured to output two different perspectives of the 3D image to the respective eyes 114 of the user 112.

In some implementations, the scope 104 is configured to communicate with one or more external devices. For example, the scope 104 is configured to transmit data to an external computing device (e.g., a mobile phone, a tablet computer, a server, etc.) and/or to receive data from the external computing device. In some implementations, the external computing device performs at least some of the analysis and/or data generation functionality described herein.

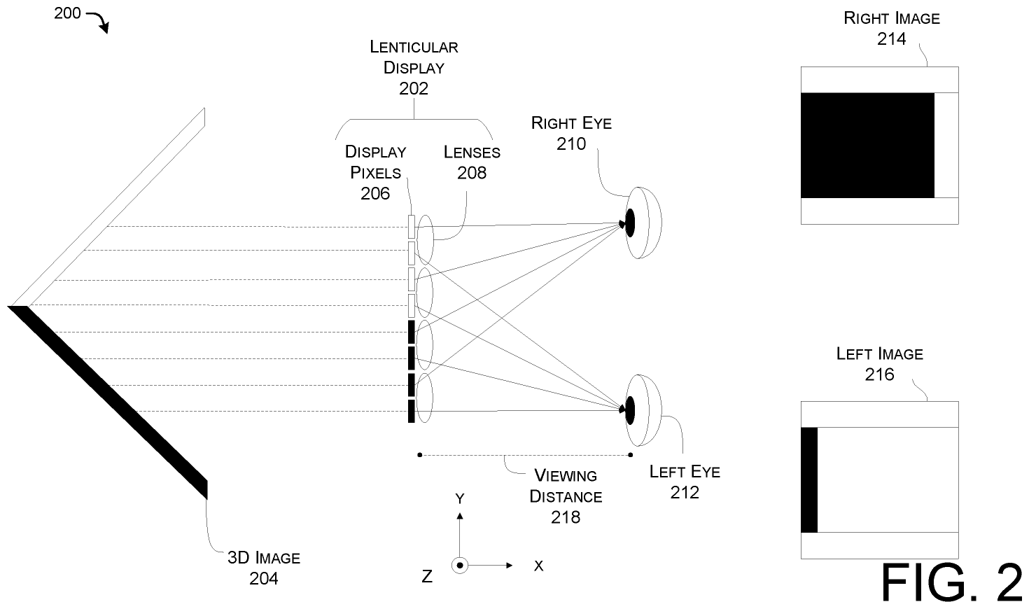

FIG. 2 illustrates an example environment 200 in which a lenticular display 202 outputs a 3D image 204 to a viewer. For instance, the lenticular display 202 may be the lenticular display 116 described above with reference to FIG. 1. In various implementations, the 3D image 204 depicts a surface of a subject, such as the interior surface of the subject's ear. For instance, the 3D image 204 may be generated by combining a 2D image of the surface with a depth image of the surface.

The lenticular display 202 includes an array of display pixels 206 and an array of lenses 208. In various implementations, the display pixels 206 are arranged as a 2D array. For instance, FIG. 2 may depict a single column of display pixels 206 extending in a y-direction, but in some cases, the lenticular display 202 includes multiple columns of the display pixels 206 arranged in a z-direction. In some cases, the display pixels 206 collectively comprise a display screen. In some implementations, a display surface of the display screen is circular. Each one of the display pixels 206 is configured to emit light. In some cases, each display pixel 206 can be controlled to emit light at a perceived frequency (i.e., color) and at a perceived intensity. For example, each display pixel 206 may be configured to output a value of an image pixel or a voxel. The display pixels 206 are configured to emit light in the x-direction. In some implementations, a collimator (not illustrated) is disposed between the display pixels 206, which may steer light emitted by the display pixels 206 in the x-direction. For example, a metal (e.g., aluminum) border is disposed between the display pixels 206.

The lenses 208, in various cases, are also arranged as a 2D array. For example, the lenticular display 202 includes multiple columns of the display pixels 206 arranged in a z-direction. In various cases, the lenses 208 are disposed between the display pixels 206 and a viewer, such as between the display pixels 206 and the right eye 210 and left eye 212 of the viewer. The lenses 208 overlap the display pixels 206 in the x-direction. For instance, the lenses 208 are configured to refract light emitted by the display pixels 206. Although FIG. 2 illustrates individual lenses 208 overlapping and/or refracting light output by multiple display pixels 206, implementations are not so limited. For example, in some cases, a single lens 208 overlaps a single display pixel 206.

Despite the display pixels 206 being arranged in a 2D display screen, the lenticular display 202 may output the 2D image 204 to the right eye 210 and the left eye 212 of the viewer based on the refraction of the lenses 208. In various cases, the lenses 208 redirect the light output by the display pixels 206 in multiple directions. Even though the display pixels 206 are configured to output light in the x-direction, the lenses 208 may steer the light in various xy-directions that intersect the right eye 210 and left eye 212. By steering the light in these different directions, the right eye 210 perceives a different 2D image than the left eye 212. That is, a right image 214 received by the right eye 210 is different than a left image 216 received by the left eye 212. The right image 214, for instance, is a projection of the 3D image 204 onto the position of the right eye 210. The left image 216, for instance, is a projection of the 3D image 206 onto the position of the left eye 212. As a result, the viewer may perceive the 3D image 204 three-dimensionally by viewing the lenticular display 202.

In various implementations, the right eye 210 and/or the left eye 212 may be separated from the lenticular display 202 by a viewing distance 218. For instance, the viewing distance 218 may be a distance between the lenses 208 and a lens of the right eye 210 or the left eye 212. In various implementations, the light output by the display pixels 206 and the lenses 208 are optimized for the viewing distance 218.

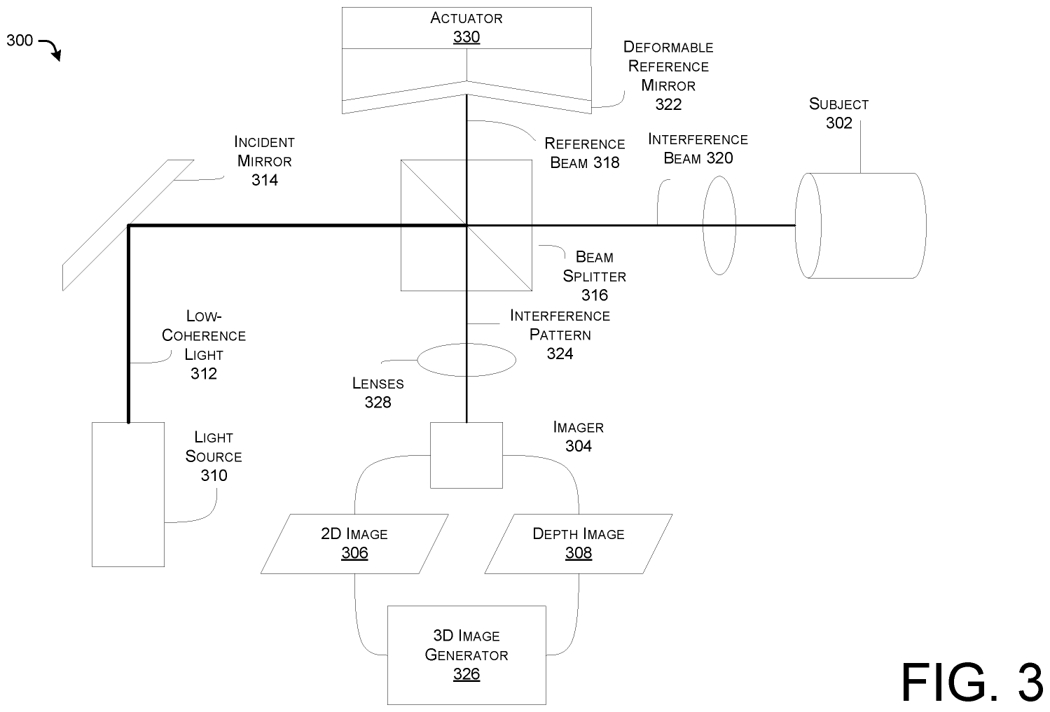

FIG. 3 illustrates an example environment 300 for generating a 3D image of a subject 302 using the same imager 304 to obtain a 2D image 306 and a depth image 308 of the subject 302. For example, the environment 300 can be at least partially integrated into the scope 104 described above with reference to FIG. 1.

In various implementations, a light source 310 emits low coherence light 312. In some implementations, the light source 310 includes one or more LEDs, a xenon-based light source, a halogen bulb, a tungsten filament, or any combination thereof configured to generate the low coherence light 312.

An incident mirror 314, in various cases, reflects the low coherence light 312 to a beam splitter 316. The incident mirror 314, for instance, is a digital micro mirror device (DMMD) or a digital light pressing (DLP) mirror. In various cases, the beam splitter 316 is configured to divide the low coherence light 312 into a reference beam 318 and an interference beam 320. The beam splitter 316 directs the reference beam 318 to a deformable reference mirror 322. The deformable reference mirror 322 reflects the reference beam 318 to the imager 304. In contrast, the beam splitter 316 directs the interference beam 320 to the subject 302. A reflection of the interference beam 320 from the subject 302 is received by the beam splitter 316, which may direct the reflection to the imager 304. Collectively, the reflection of the reference beam 318 from the deformable reference mirror 322 and the reflection of the interference beam 320 generate an interference pattern 324 that is detected by the imager 304.

In various implementations, the imager 304 includes an array of photosensors configured to detect the interference pattern 324. In various implementations, the imager 304 is configured to generate the depth image 308 based on the interference pattern 324.

Further, the imager 304 may detect at least a portion of the reflection of the interference beam 320 from the subject 302. Based on the resulting image of this reflection, the imager 304 may generate a 2D image 306 of the subject 302. For example, based on the intensity and/or frequency of the reflection of the interference beam 320 detected by the imager 304, the imager 304 may generate a 2D image of the subject 302. The 2D image 306 may depict the subject 302 from a single perspective.

In various implementations, a 3D image generator 326 generates a 3D image of the subject 302 based on the 2D image 306 and the depth image 308. According to some cases, the 3D image generator 326 is implemented by one or more processors configured to execute instructions. For instance, the 3D image generator 326 is configured to generate an array of voxels representing the subject 302 in 3D space. In various cases, the 3D image generator 326 performs ray tracing, shading, and tessellation to generate the 3D image of the subject 302. In various implementations, the 3D image generator 326 further performs point-of-view (POV) display segmentation to encode the 3D image for display via a lenticular display.

In some cases, the environment 300 includes one or more lenses 328 configured to refract low-coherence light 312, the reference beam 318, the reflection of the reference beam 318 from the deformable reference mirror 322, the interference beam 320, the reflection of the interference beam 320 from the subject 302, or any combination thereof. For example, the lens(es) 328, in various cases, are disposed between the light source 310 and the incident mirror 314, between the beam splitter 316 and the subject 302, between the beam splitter 316 and the deformable reference mirror 322, between the beam splitter 316 and the imager 304, or any combination thereof. In some cases, the position(s) of the lens(es) 328 can be modified via one or more actuators (not illustrated), such as in order to adjust the focus and/or resolution of the 2D image 306 and/or the depth image 308.

According to various implementations, the shape of a reflective surface of the deformable reference mirror 322 is adjustable. For example, an actuator 330 is physically coupled to the deformable reference mirror 322. In various cases, the actuator 330 is configured to alter a curvature and/or position of the deformable reference mirror 322. For example, if the deformable reference mirror 322 is in a concave shape, the reflection of the reference beam 318 converges. Alternatively, if the deformable reference mirror 322 is in a convex shape, the reflection of the reference beam 318 diverges.

In various implementations, the curvature of the deformable reference mirror 322 is optimized according to the interference pattern 324. In various cases, the curvature of the deformable reference mirror 322 corresponds to a depth of the subject 302 represented by the interference pattern 324. For example, if the reference beam 318 and the interference beam 320 are too mismatched, the interference pattern 324 cannot be adequately converted into the depth image 308. Changing the curvature of the deformable reference mirror 322 can correct the depth alignment of the interference pattern 324.

In various implementations, the light source 310 includes at least two elements configured to output different types of light. One element may be configured to output the low-coherence light 312 or even coherent light, which may be optimized for generating the interference pattern 324 and the depth image 308. Another element may be configured to output a more broad-spectrum light, such as white light, that is configured to illuminate the subject 302 in order to obtain the 2D image 306. In some implementations, the light source 310 includes a light-emitting element and a filter wheel that selectively passes coherent or low-coherence light (e.g., when obtaining the depth image 308) and emits white light (e.g., when obtaining the 2D image 306) at different times.

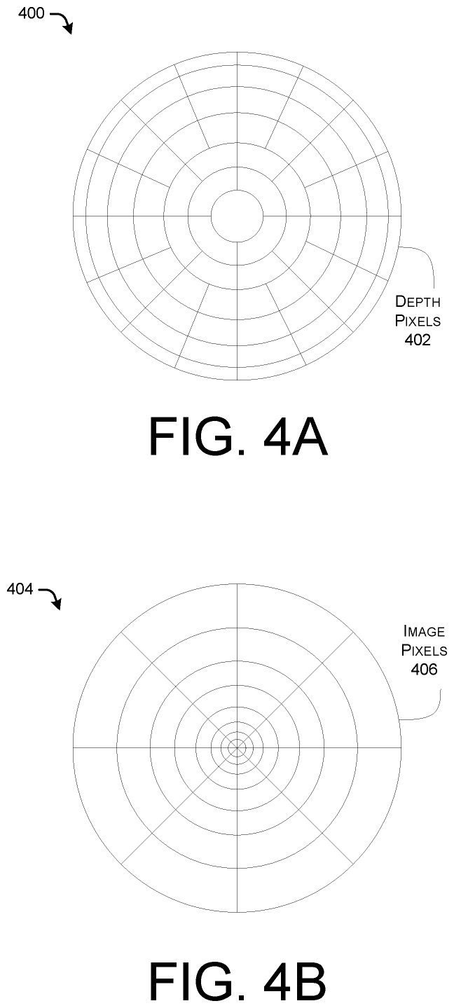

FIGS. 4A and 4B illustrate example resolutions of images generated by a depth rendering scope. FIG. 4A, for example, illustrates a depth image 400 including various depth pixels 402. Each depth pixel 402 represents a distance between a surface and an imager at a particular angle with respect to a reference ray extending from a center of the imager. For example, the depth pixels 402 along the edge of the depth image 404 represent distances between the surface and the imager at an edge of the field-of-view (FOV) of the imager.

As shown, the depth pixels 402 have a higher resolution at a periphery of the depth image 400, and may have a lower resolution at a center of the depth image 400. For example, the depth image 400 may depict the center of the FOV with a lower pixel density than a periphery of the FOV. In various cases, this resolution distribution may be suitable for imaging the interior surface of an orifice, such as an ear. Because a center of the FOV of an orifice is a hole with relatively high depth and minimal diagnostic value, the depth image 400 may be generated to have relatively high resolution depicting a periphery of the FOV and relatively low resolution depicting a center of the FOV.

FIG. 4B illustrates a 2D image 404 including various image pixels 406. Each projection pixel 406, for example, represents an intensity and/or frequency of light reflected an individual region of the surface. The image pixels 406 have a higher resolution at the center of the image 404 and a lower resolution at the periphery of the image 404. For example, the image 404 may depict the periphery of the FOV with a lower pixel density than the center of the FOV. In various cases, the periphery of the FOV of an orifice is the edge of a hole that may be difficult to focus on and/or depict with a image. Accordingly, the periphery of the image 404 may have less diagnostic value than the center of the image 404.

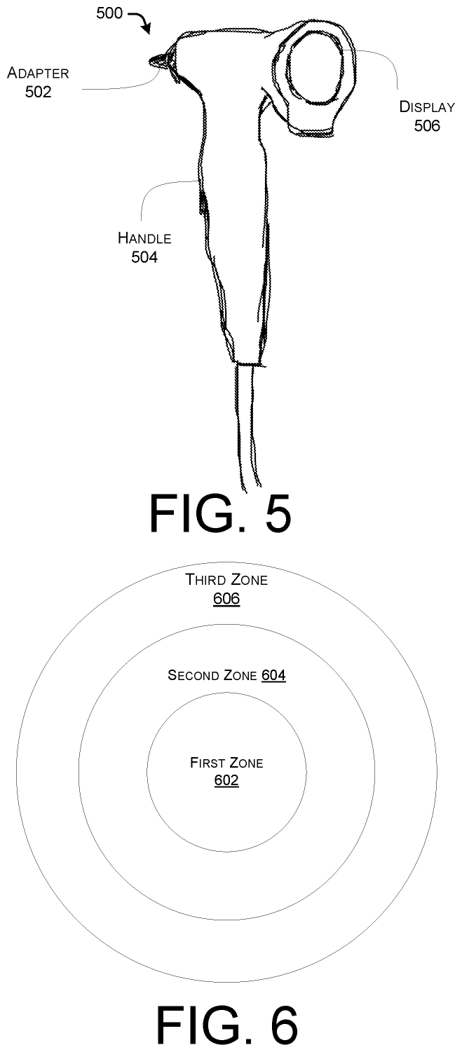

FIG. 5 illustrates an example scope 500 configured to obtain and display a 3D image of a subject. For example, the scope 500 may be the scope 104 described above with reference to FIG. 1.

In various implementations, the scope 500 includes an adapter 502 configured to be inserted into an orifice of a subject, such as an ear canal. In some cases, the adapter 502 is disposable and removably coupled to the rest of the scope 500. The scope 500 includes a handle 504 configured to be grasped and held by the hand of a user, such as a clinician operating the scope 500. Further, the scope 500 includes a display 506. The display 506, in some cases, is circular. In various implementations, the display 506 is a lenticular display. For example, the display 506 includes a single 2D screen that outputs the 3D image via an array of lenses overlapping the 2D screen.

FIG. 6 illustrates an example lenticular display 600 divided into three radial zones. In particular, the display 600 includes a first zone 602 that includes a center of the lenticular display 600, a second zone 604 disposed radially from the first zone 602, and a third zone 606 extending from a radial edge of the lenticular display 600. The second zone 604, for instance, is disposed between the first zone 602 and the third zone 606. The lenticular display 600 is configured to visually present a 3D image generated based on a 2D image and a depth image of a surface. In various implementations, the first zone 602 outputs a first portion of the 3D image, which is generated based on a first portion of the 2D image and a first portion of the depth image. The first portion of the 2D image includes relatively high spatial resolution, whereas the first portion of the depth image has relatively low spatial resolution. The third zone 606 outputs a third portion of the 3D image, which is generated based on a third portion of the 2D image and a third portion of the depth image. The third portion of the depth image includes relatively high spatial resolution, whereas the third portion of the 2D image has relatively low spatial resolution. The second zone 604 outputs a second portion of the 3D image, which is generated based on a second portion of the 2D image and a second portion of the depth image. The second portion of the 2D image may have a moderate spatial resolution compared to the first portion and the third portion of the 2D image. Similarly, the second portion of the depth image may have a moderate spatial resolution compared to the first portion and the third portion of the depth image.

In various implementations, the display 600 has a uniform resolution (e.g., each display pixel in the display 600 has a consistent width and height). The resolution of the display pixels in the display 600 corresponds to a maximum resolution of the FOV. However, in cases where the display 600 is a lenticular display, a single location in the depicted image may be represented by multiple display pixels, which can lower the image resolution. Thus, as more depth information is portrayed by the display 600, the less image resolution is portrayed by the display 600. In various cases, the first zone 602 depicts the 3D image at a high image resolution, with minimal depth information. In contrast, the third zone 606 depicts the 3D image at low image resolution but with high depth information.

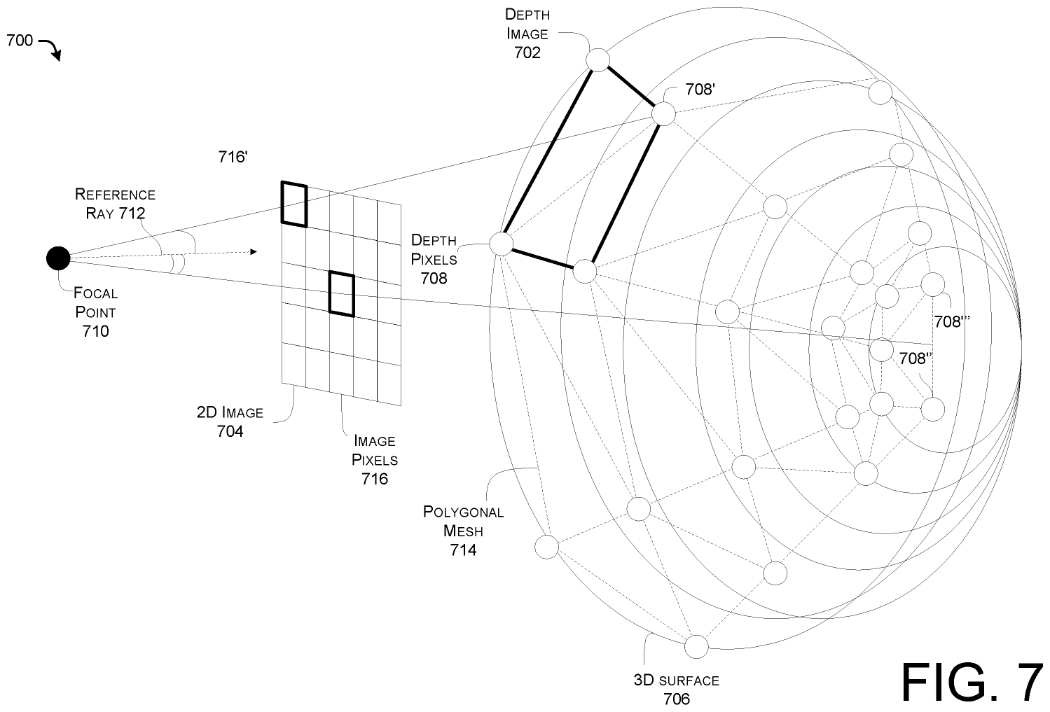

FIG. 7 illustrates an example of a depth image 702 and a 2D image 704 being combined to generate a 3D image of a surface. In various implementations, the depth image 702 is defined according to multiple depth pixels 708 that define the depth image 702 in 3D space. Each of the depth pixels 708, for instance, is defined according to a distance to a focal point 710 and an angle between a ray extending from the focal point 710. In particular examples, a first depth pixel 708′ is defined as a first distance from the focal point 710 at a first angle with respect to a reference ray 712. A second depth pixel 708″ is defined as a second distance from the focal point 710 at a second angle with respect to the reference ray 712. Collectively, the depth pixels 708 represent detected positions along a surface that is being imaged. The focal point 710, for instance, is located at one or more imagers that have generated the depth image 702 and/or the 2D image 704. In various cases, the depth pixels 708 of the depth image 702 are defined in a radial or polar coordinate system with respect to the focal point 710. In some cases, the depth pixels 708 of the depth image 702 are defined using positions along three linear dimensions (e.g., an x dimension, a y dimension, and a z dimension).

In various implementations, the depth pixels 708 are used to define a polygonal mesh 714. For example, the depth pixels 708, in some cases, define vertices of the polygonal mesh 714. Alternatively, each depth pixel 708 defines a face (e.g., a center of the face) of the polygonal mesh 714. In the example illustrated in FIG. 7, the polygonal mesh 714 is defined as triangles extending between the depth pixels 708 of the depth image 702. However, in some cases, the polygonal mesh 714 is defined as a mesh of other types of polygonal shapes. Although the example of FIG. 7 illustrates only a handful of depth pixels 708, it can be understood that additional depth pixels 708 may also be defined in the 3D space.

The 2D image 704, in various implementations, includes an array of image pixels 716. In various implementations, the image pixels 716 are defined according to the intensity and/or frequency of light reflected from positions along the surface being imaged. In various cases, the 2D image 704 can be defined along a plane that is parallel to a sensing face of the imager(s). For example, if the imager(s) include a 2D photosensor array defined in a plane along a first direction and a second direction, then the 2D image 704 may be defined along a parallel plane.

In various implementations, the 3D image of the surface is generated by ray tracing. For example, rays may be defined from the focal point 710 through the image pixels 716 and onto the faces of the polygonal mesh 714 defined by the depth pixels 708. Thus, the image pixels 716 are projected onto the polygonal mesh 714. In various implementations, voxels in the 3D surface 706 are defined based on nearby and/or overlapping vertices of the depth image 702 and/or values of the image pixels 716 that project near the voxels. In some cases, a first example voxel in the 3D surface 706 is located at a position in 3D space that overlaps the first depth pixel 708′. Thus, the first example voxel is located along the surface being imaged. The value of the first example voxel (e.g., a color of the first example voxel) is determined based on a value of a first image pixel 716′, which is projected onto the first depth pixel 708′ and the first example voxel in the 3D surface 706. For instance, the projection of the first image pixel 716′ is directly onto the first example voxel, such that the value of the first example voxel is equivalent to the value of the first image pixel 716′. the vertices of the polygonal mesh 714 are defined based on the values of the image pixels 716.

According to some cases, not all of the image pixels 716 project perfectly onto the positions corresponding to the depth pixels 708. For example, a second image pixel 716″ projects onto a position between a second depth pixel 708″ and a third depth pixel 708″′ in the depth image 702. In various implementations, the value of the second image pixel 716″ is used to define the value of a voxel located between the second depth pixel 708″ and the third depth pixel 708″′ in the 3D space.

In various implementations, the spatial resolution of the image pixels 716 may be different than that of the depth pixels 708 at various regions within 3D space. In some cases, a value of an example face of the polygonal mesh 714 is defined by linearly interpolating at least two of the image pixels 716. For example, if two neighboring image pixels 716 project onto two separate voxels of the polygonal mesh 714, wherein another voxel is disposed between the two separate voxels of the polygonal mesh 714, then a value of the middle voxel can be defined based on a combination of the values of the two neighboring image pixels 716. For instance, the combination may be a linear interpolation of the values of the two neighboring image pixels 716. For instance, the combination may be a mean of the values.

By projecting the image pixels 716 onto the polygonal mesh 714, the voxels of the 3D surface 706 may be defined. In various implementations, the 3D surface 706 is generated by performing tessellation on the polygonal mesh 714. For example, vertices are added to the polygonal mesh 714 in order to smooth the polygonal mesh 714 into the 3D surface 706. For example, the polygonal mesh 714 is converted into the 3D surface 706 using at least one tessellation algorithm. In some cases, the 3D surface 706 is defined as a polygonal shape with a greater number of faces than the polygonal mesh 714. In various implementations, the projection of the image pixels 716 is performed after the tessellation, such that the projection is performed on voxels defining the 3D surface 706 rather than the depth pixels 708. However, implementations are not so limited.

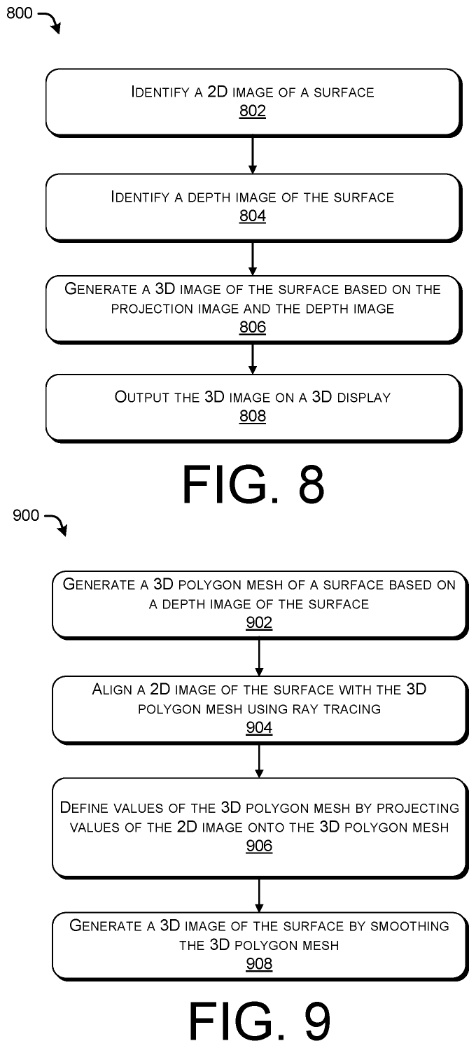

FIG. 8 illustrates an example process 800 for providing a 3D image of a surface. In various implementations, the process 800 is performed by an entity, such as a scope (e.g., the scope 104 described above with reference to FIG. 1), at least one server, at least one computing device, at least one processor, or a combination thereof.

At 802, the entity identifies a 2D image of a surface. For example, the entity receives and/or generates the 2D image that is indicative of light reflected from the surface. In some cases, the 2D image indicates the intensity and/or color of the light reflected from the surface. In various implementations, the entity includes an imager configured to generate the 2D image of the surface. For instance, the entity may emit light onto the surface, detect the light reflected from the surface, and generate the 2D image based on the detected light. The 2D image may be represented by multiple image pixels. According to various cases, a spatial resolution of the 2D image decreases radially from a center of the FOV of the 2D image.

At 804, the entity identifies a depth image of the surface. In various implementations, the entity receives and/or generates data indicative of a distance between a sensor and the surface. In some cases, the entity generates the depth image using ultrasound and/or OCT imaging. For instance, the entity may detect an interference pattern using the same imager used to detect the 2D image. According to various cases, a spatial resolution of the depth image increases radially from a center of the FOV of the depth image. In some cases, the depth image has a different relative spatial resolution than the 2D image.

At 806, the entity generates a 3D image of the surface based on the image and the depth image. In various implementations, the entity defines locations along the surface in 3D space using the depth image. In some cases, the entity generates a 3D binary image, wherein the nonzero values of the voxels in the 3D binary image correspond to the locations along the surface. For instance, the entity converts the radial data in the depth image to 3D coordinates. In some cases, voxels disposed between neighboring depths in the depth image are defined by geometrically interpolating the neighboring depths in 3D space.

The entity, in some cases, colorizes and/or texturizes the 3D binary image using the 2D image. For example, the entity defines values of the nonzero voxels based on the image pixels in the 2D image of the surface. In various implementations, the entity generates the 3D image by ray tracing the values of the image pixels in the 2D image onto the shape of the surface (e.g., as defined in the 3D binary image). In some cases, the entity performs linear interpolation to define the values of one or more nonzero voxels disposed between rays traced between neighboring image pixels in the 2D image and projected onto the nonzero values of the 3D binary image. In some implementations, the entity performs tessellation to generate the 3D image.

At 808, the entity outputs the 3D image on a 3D display. In some implementations, the entity outputs the 3D image via a lenticular display. For instance, the entity controls a single 2D display pixel array and/or an array of lenses overlapping the pixel array to direct the 3D image in two directions corresponding to two eyes of a user. For instance, the entity causes the apparent image perceived by a first eye of the user to be a 2D projection of the 3D image from a first angle, and the apparent image perceived by a second eye of the user to be a 2D projection of the 3D image from a second angle, such that the perceived images are in parallax. In various implementations, the parallax causes the user to perceive the 2D projections in three dimensions. Accordingly, the entity can visually output the 3D image to a user using a single 2D array of display pixels. In some implementations, the entity outputs the 2D projections onto two separate displays that are respectively viewed by the eyes of the user.

FIG. 9 illustrates an example process 900 for generating a 3D image of a surface. In various implementations, the process 900 is performed by an entity, such as a scope (e.g., the scope 104 described above with reference to FIG. 1), at least one server, at least one computing device, at least one processor, or a combination thereof.

At 902, the entity generates a 3D polygon mesh of a surface based on a depth image of the surface. In various implementations, the depth pixels define a surface in the 3D space. Various polygons are defined in the 3D space, wherein the depth pixels are located at vertices of the polygons. Thus, the surface is approximated as a collection of flat polygonal surfaces (e.g., in a polygonal mesh). In various implementations, the 3D polygon mesh is defined as a binary 3D image in a 3D space. Therefore, nonzero voxels in the binary 3D image are assigned at the defined surface. Zero value voxels are defined elsewhere in the binary 3D image.

At 904, the entity aligns a 2D image of the surface with the 3D polygon mesh using ray tracing. At 906, the entity defines values of the 3D polygon mesh by projecting values of the 2D image onto the 3D polygon mesh. In various implementations, values of individual image pixels in the 2D image are projected onto polygon mesh. In various cases, if neighboring image pixels of the 2D image project onto the 3D polygon mesh at different locations that are separated by a vertex of the 3D polygon mesh, the value (e.g., color) of the vertex of the 3D polygon mesh is defined based on a linear interpolation of the values of the projected image pixels.

At 908, the entity generates a 3D image of the surface by smoothing the 3D polygon mesh. In various implementations, tessellation is performed on the colorized 3D polygon mesh in order to smooth the surface. The resultant smoothed image is defined as a 3D image of the surface.

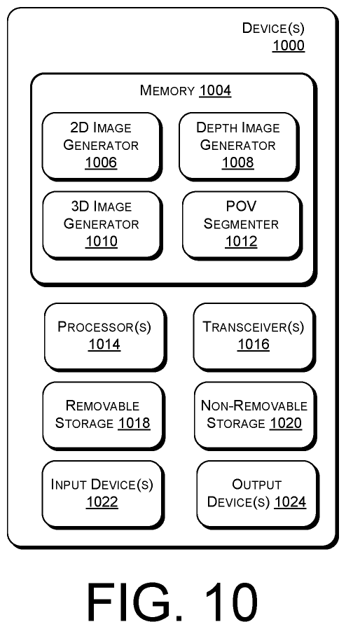

FIG. 10 illustrates at least one example device 1000 configured to enable and/or perform the some or all of the functionality discussed herein. Further, the device(s) 1000 can be implemented as one or more server computers 1002, a network element on a dedicated hardware, as a software instance running on a dedicated hardware, or as a virtualized function instantiated on an appropriate platform, such as a cloud infrastructure, and the like. It is to be understood in the context of this disclosure that the device(s) 1000 can be implemented as a single device or as a plurality of devices with components and data distributed among them.

As illustrated, the device(s) 1000 comprise a memory 1004. In various embodiments, the memory 1004 is volatile (including a component such as Random Access Memory (RAM)), non-volatile (including a component such as Read Only Memory (ROM), flash memory, etc.) or some combination of the two.

The memory 1004 may include various components, such as at least one 2D image generator 1006, at least one depth image generator 1008, a 3D image generator 1010, a POV segmenter 1012, and the like. Any of the 2D image generator 1006, the depth image generator 1008, the 3D image generator 1010, and the POV segmenter 1012 can comprise methods, threads, processes, applications, or any other sort of executable instructions. The 2D image generator 1006, the depth image generator 1008, the 3D image generator 1010, and the POV segmenter 1012 and various other elements stored in the memory 1004 can also include files and databases. In various cases, the 2D image generator 1006 includes instructions for generating a 2D image based on light detected by an image sensor. The depth image generator 1008, in various cases, includes instructions for generating a depth image based on an interference pattern detected by an image sensor and/or ultrasound detected by a transducer. In various implementations, the 3D image generator 1010 includes instructions for generating a 3D image based on a 2D image and a depth image of the same scene. In some examples, the POV segmenter 1012 includes instructions for outputting the 3D image on a lenticular display.

The memory 1004 may include various instructions (e.g., instructions in the 2D image generator 1006, depth image generator 1008, 3D image generator 1010, and/or POV segmenter 1012), which can be executed by at least one processor 1014 to perform operations. In some embodiments, the processor(s) 1014 includes a Central Processing Unit (CPU), a Graphics Processing Unit (GPU), or both CPU and GPU, or other processing unit or component known in the art.

The device(s) 1000 can also include additional data storage devices (removable and/or non-removable) such as, for example, magnetic disks, optical disks, or tape. Such additional storage is illustrated in FIG. 10 by removable storage 1018 and non-removable storage 1020. Tangible computer-readable media can include volatile and nonvolatile, removable and non-removable media implemented in any method or technology for storage of information, such as computer readable instructions, data structures, program modules, or other data. The memory 1004, removable storage 1018, and non-removable storage 1020 are all examples of computer-readable storage media. Computer-readable storage media include, but are not limited to, RAM, ROM, EEPROM, flash memory or other memory technology, CD-ROM, Digital Versatile Discs (DVDs), Content-Addressable Memory (CAM), or other optical storage, magnetic cassettes, magnetic tape, magnetic disk storage or other magnetic storage devices, or any other medium which can be used to store the desired information and which can be accessed by the device(s) 1000. Any such tangible computer-readable media can be part of the device(s) 1000.

The device(s) 1000 also can include input device(s) 1022, such as a keypad, a cursor control, a touch-sensitive display, voice input device, etc., and output device(s) 1024 such as a display, speakers, printers, etc. In particular implementations, a user can provide input to the device(s) 1000 via a user interface associated with the input device(s) 1022 and/or the output device(s) 1024. In various implementations, the input device(s) 1022 include a distance sensor configured to detect a distance between a user and the device(s) 1000, an ultrasound transducer, an OCT imaging system, or a combination thereof. According to various cases, the input device(s) 1022 include a single imager. In various implementations, the output device(s) 1024 include a lenticular display.

As illustrated in FIG. 10, the device(s) 1000 can also include one or more wired or wireless transceiver(s) 1016. For example, the transceiver(s) 1016 can include a Network Interface Card (NIC), a network adapter, a LAN adapter, or a physical, virtual, or logical address to connect to the various base stations or networks contemplated herein, for example, or the various user devices and servers. To increase throughput when exchanging wireless data, the transceiver(s) 1016 can utilize Multiple-Input/Multiple-Output (MIMO) technology. The transceiver(s) 1016 can include any sort of wireless transceivers capable of engaging in wireless, Radio Frequency (RF) communication. The transceiver(s) 1016 can also include other wireless modems, such as a modem for engaging in Wi-Fi, WiMAX, Bluetooth, or infrared communication.

In some implementations, the transceiver(s) 1016 can be used to communicate between various functions, components, modules, or the like, that are comprised in the device(s) 1000. For instance, the transceivers 1016 may facilitate communications between the 2D image generator 1006, the depth image generator 1008, the 3D image generator 1010, and/or the POV segmenter 1012.

In some instances, one or more components may be referred to herein as “configured to,” “configurable to,” “operable/operative to,” “adapted/adaptable,” “able to,” “conformable/conformed to,” etc. Those skilled in the art will recognize that such terms (e.g., “configured to”) can generally encompass active-state components and/or inactive-state components and/or standby-state components, unless context requires otherwise.

As used herein, the term “based on” can be used synonymously with “based, at least in part, on” and “based at least partly on.”

As used herein, the terms “comprises/comprising/comprised” and “includes/including/included,” and their equivalents, can be used interchangeably. An apparatus, system, or method that “comprises A, B, and C” includes A, B, and C, but also can include other components (e.g., D) as well. That is, the apparatus, system, or method is not limited to components A, B, and C.

Although the subject matter has been described in language specific to structural features and/or methodological acts, it is to be understood that the subject matter defined in the appended claims is not necessarily limited to the specific features or acts described.

What is claimed is:

1. An otoscope, comprising:
a first light source configured to emit low-coherence light;
a beam splitter configured to split the low-coherence light into a first beam and a second beam;
a lens assembly configured to transmit the first beam to an ear canal and to receive a reflection of the first beam from the ear canal;
a reference mirror configured to emit a reflection of the second beam by reflecting the second beam;
an imager configured to receive an interference pattern comprising the reflection of the first beam and the reflection of the second beam, to generate depth data based on the interference pattern, to receive a reflection of the low-coherence light from the ear canal, and to generate 2D image data based on the reflection of the low-coherence light;
a lenticular display comprising:
a screen comprising an array of display pixels;
an array of lenses, an example lens in the array of lenses being overlaid with a group of display pixels among the array of display pixels, the example lens refracting light emitted by a first pixel in the group of display pixels in a first direction and refracting light emitted by a second pixel in the group of display pixels in a second direction;
a processor; and
memory storing instructions that, when executed by the processor, cause the processor to perform operations comprising:
generating a three-dimensional (3D) image of the ear canal based on the depth data and the 2D image data; and
causing the lenticular display to visually output the 3D image of the ear canal.

2. The otoscope of claim 1, further comprising:
a housing comprising a handle; and
at least one battery configured to output power to the imager, the lenticular display, the processor.

3. The otoscope of claim 1, further comprising:
a distance sensor configured to detect a first viewing distance between the otoscope and a user at a first time and to detect a second viewing distance between the otoscope and the user at a second time,
wherein:
the operations further comprise:
determining that the first viewing distance is outside of a predetermined range;
outputting an alert based on determining that the first viewing distance is outside of the predetermined range; and
determining that the second viewing distance is within the predetermined range, and
causing the lenticular display to visually output the 3D image of the ear canal is based on determining that the second viewing distance is within the predetermined range.

4. A scope, comprising:
at least one imager configured to:
generate depth data by detecting a distance between the at least one imager and a surface; and
generate 2D image data by detecting light reflected from the surface;
a three-dimensional (3D) display;
a processor; and
memory storing instructions that, when executed by the processor, cause the processor to perform operations comprising:
generate a 3D image of the surface by combining the depth data and the 2D image data; and
cause the 3D display to output the 3D image of the surface.

5. The scope of claim 4, wherein the at least one imager comprises:
a light source configured to emit low-coherence light;
a beam splitter configured to split the low-coherence light into a first beam and a second beam, the beam splitter emitting the first beam toward the surface;
a reference mirror configured to emit a reflection of the second beam by reflecting the second beam; and
an image sensor array configured to generate the depth data by detecting a coherence image comprising a reflection of the first beam from the surface and the reflection of the second beam.

6. The scope of claim 5, further comprising:
an input device configured to detect an input signal from a user; and
an actuator configured to alter a curvature of the reference mirror, the reference mirror being deformable,
wherein the operations further comprise causing the actuator to alter the curvature of the reference mirror based on the input signal.

7. The scope of claim 5, further comprising:
at least one lens configured to refract at least one of the low-coherence light, the first beam, the second beam, the reflection of the first beam, or the reflection of the second beam.

8. The scope of claim 5, the light source being a first light source, wherein:
the image sensor array is further configured to generate the 2D image data by detecting a image comprising a reflection of the low-coherence light from the surface.

9. The scope of claim 4, wherein the 3D display comprises:
a screen comprising an array of pixels; and
an array of lenses, an example lens in the array of lenses overlapping a group of pixels in the array of pixels, the example lens directing light emitted by a first pixel in the group of pixels in a first direction and directing light emitted by a second pixel in the group of pixels in a second direction.

10. The scope of claim 4, wherein a spatial resolution of the depth data is greater at a periphery of a field-of-view of the depth data than a spatial resolution of the depth data at a center of the field-of-view, and
wherein a spatial resolution of the 2D image data is greater at a center of a field-of-view of the 2D image data than a spatial resolution of the 2D image data at a periphery of the field-of-view.

11. The scope of claim 4, further comprising:
a sensor configured to detect a distance between the scope and a user,
wherein the operations further comprise:
causing the 3D display to visually output a recommendation to increase or decrease the distance.

12. The scope of claim 4, further comprising:
a sensor configured to detect a distance between the scope and a user,
wherein the operations further comprise:
adjusting a viewing distance of the 3D image being visually presented by the 3D display based on the distance between the scope and the user.

13. A method, comprising:
generating depth data by detecting a distance between at least one imager and a surface;
generating 2D image data by detecting light reflected from the surface;
generating a 3D image of the surface by combining the depth data and the 2D image data; and
outputting the 3D image of the surface.

14. The method of claim 13, wherein detecting the distance between the at least one imager and the surface comprises detecting ultrasound reflected from the surface or detecting an interference pattern reflected from the surface.

15. The method of claim 13, wherein outputting the 3D image of the surface comprises:
generating a first 2D projection of the 3D image from a first direction;
generating a second 2D projection of the 3D image from a second direction;
displaying, on a first screen, the first 2D projection; and
displaying, on a second screen, the second 2D projection.

16. The method of claim 13, wherein outputting the 3D image of the surface comprises:
displaying, on a lenticular display, the 3D image.

17. The method of claim 13, wherein generating the 3D image of the surface by combining the depth data and the 2D image data comprises:
identifying a depth in the depth data;
identifying at least one first value in the 2D image data corresponding to the depth in the depth data; and
defining a second value of a point in the 3D image based on the at least one first value, a location of the second value being based on the depth.

18. The method of claim 17, wherein:
identifying at least one first value in the 2D image data comprises identifying multiple first values in the 2D image data corresponding to the depth in the depth data, and
the second value comprises a mean of the multiple first values.

19. The method of claim 17, wherein generating the 3D image of the surface by combining the depth data and the 2D image data comprises:
identifying a first depth in the depth data;
defining a first point in the 3D image based on at least one first value in the 2D image data that corresponds to the first depth;
identifying a second depth in the depth data;
defining a second point in the 3D image based on at least one second value in the 2D image data that corresponds to the second depth; and
defining a third point in the 3D image by interpolating the first point and the second point.

20. The method of claim 13, wherein a spatial resolution of the depth data is greater at a periphery of a field-of-view of the depth data than a spatial resolution of the depth data at a center of the field-of-view, and
wherein a spatial resolution of the 2D image data is greater at a center of a field-of-view of the 2D image data than a spatial resolution of the 2D image data at a periphery of the field-of-view.
